(19) United States
(12) Patent Application Publication
(10) Pub. No.: US 2023/0310894 A1
(43) Pub. Date: Oct. 5, 2023

(54) RADIATION THERAPY DEVICES, PHOTOFLASH THERAPY SYSTEMS, AND ULTRA-HIGH ENERGY ELECTRON FLASH THERAPY SYSTEMS

(71) Applicant: SHANGHAI UNITED IMAGING HEALTHCARE CO., LTD., Shanghai (CN)

(72) Inventors: Peng WANG, Shanghai (CN); Cheng NI, Shanghai (CN); Shoubo HE, Shanghai (CN); Feichao FU, Shanghai (CN); Yanfang LIU, Shanghai (CN); Wei HAN, Shanghai (CN); Li WANG, Shanghai (CN)

(73) Assignee: SHANGHAI UNITED IMAGING HEALTHCARE CO., LTD., Shanghai (CN)

(21) Appl. No.: 18/194,608

(22) Filed: Mar. 31, 2023

(30) Foreign Application Priority Data
Mar. 31, 2022 (CN) 202210333130.1

(51) Int. Cl. A61N 5/10; G21K 1/093

(52) U.S. Cl. CPC A61N 5/1043; A61N 5/1045; A61N 5/1084; G21K 1/093; A61N 2005/1089

(57) ABSTRACT

The embodiments of the present disclosure provide a radiation therapy device. The radiation therapy device may comprise a beam generating device, a scanning magnet, and one or more focusing magnets. The beam generating device may be configured to generate a charged particle beam. The scanning magnet may be configured to diverge the charged particle beam. The one or more focusing magnets may be configured to deflect the charged particle beam diverged by the scanning magnet.

### CROSS-REFERENCE TO RELATED APPLICATIONS

This application claims priority to Chinese Patent Application No. 202210333130.1, filed on Mar. 31, 2022, the contents of which are hereby incorporated by reference to its entirety.

### TECHNICAL FIELD

The present disclosure relates to the field of medical device technology, and in particular, to a radiation therapy device, a photoflash therapy system, and an ultra-high energy electron flash therapy system.

### BACKGROUND

Currently, malignant tumors may be treated by using high-energy accelerated particle beam irradiation. When irradiating a particle beam to an object, energy (i.e., irradiation doses) may be given to the object along a trajectory of the particle beam in the object. In the case of concentrated irradiation doses to a restricted region (i.e., a focus) inside the object, the charged particle beam may be irradiated from all directions in a way that the particle beam coincides with the focus, which can improve the concentration of the irradiation doses. One of common manners is placing the particle beam or particle source on a frame that rotates around the object, which needs a relatively large rotating mechanism and relatively large working space; another common manner is placing multiple acceleration devices with different angles around the object, which needs a relatively high cost because of the multiple devices and relatively large footprint. Therefore, it is desirable to provide a radiation therapy device with a relatively small size and low cost.

### SUMMARY

One aspect of the present disclosure provides a radiation therapy device, comprising a beam generating device, a scanning magnet, and one or more focusing magnets. The beam generating device may be configured to generate a charged particle beam, the scanning magnet may be configured to diverge the charged particle beam, and the one or more focusing magnets may be configured to deflect the charged particle beam diverged by the scanning magnet.

In some embodiments, each focusing magnet may include an entrance and an exit, wherein the entrance may be configured for injection of the charged particle beam, and the exit may be configured for emission of the charged particle beam.

In some embodiments, the charged particle beam deflected by the focusing magnets may converge at a treatment center point within a range where the treatment center point is taken as a center and a central angle may be greater than 180°.

In some embodiments, the radiation therapy device may further include one or more targets, and each target may be arranged at an exit of each focusing magnet.

In some embodiments, the charged particle beam may impact the target to generate a photon beam within a range where the treatment center point is taken as a center and a central angle is greater than 180°.

In some embodiments, the radiation therapy device may further include a multi-leaf collimator, and the multi-leaf collimator may be arc and arranged at the exit of each focusing magnet.

In some embodiments, the exit of each focusing magnet may be provided with at least one treatment piece, and the at least one treatment piece may be movably arranged at the exit of each focusing magnet.

In some embodiments, a count of the focusing magnets may be at least two, the at least two focusing magnets may be arranged adjacently or oppositely; and when the at least two focusing magnets are arranged oppositely, the exits of the at least two focusing magnets may be opposite.

In some embodiments, a deflection angle of the charged particle beam may be within a range of 0-150°.

In some embodiments, each focusing magnet may bend towards the exit of each focusing magnet, and the exit of each focusing magnet may be arc.

In some embodiments, a magnetic field intensity of a magnetic field generated by the focusing magnets may be not uniformly distributed.

In some embodiments, a length of each focusing magnet may be less than or equal to 4 m; and a width of each focusing magnet may be less than or equal to 2 m.

In some embodiments, each focusing magnet may include a first portion and a second portion, a gap may be arranged between the first portion and the second portion; and a dimension of a middle position of the gap may be greater than a dimension of two ends position of the gap in a vertical direction.

In some embodiments, a difference between the dimension of the middle position of the gap and the dimension of the two ends position of the gap may be less than or equal to 20 cm.

In some embodiments, a ratio of the dimension of the middle position of the gap to the dimension of the two ends position of the gap may be less than or equal to 5:1.

In some embodiments, a dimension of the gap may gradually decrease from the middle position to the two ends position.

In some embodiments, a trajectory of the gap from the middle position to the two ends position may be a straight line.

In some embodiments, a trajectory of the gap from the middle position to the two ends position may be an arc.

Another aspect of the present disclosure provides a photoflash therapy system, including a radiation therapy device. The radiation therapy device may include a beam generating device, a scanning magnet, and one or more focusing magnets. The beam generating device may be configured to generate a charged particle beam; the scanning magnet may be configured to diverge the charged particle beam; and the one or more focusing magnets may be configured to deflect the charged particle beam diverged by the scanning magnet. The photoflash therapy system may further comprise a target and a multi-leaf collimator. The beam generating device may include at least one of a petal-shaped accelerator and a cyclotron.

Another aspect of the present disclosure provides an ultra-high energy electron flash therapy system, including a radiation therapy device. The radiation therapy device may include a beam generating device, a scanning magnet, and one or more focusing magnets. The beam generating device may be configured to generate a charged particle beam; the scanning magnet may be configured to diverge the charged particle beam; and the one or more focusing magnets may be configured to deflect the charged particle beam diverged by the scanning magnet. The ultra-high energy electron flash therapy system may further comprise at least one treatment piece. The beam generating device may include at least one of a high-gradient radio frequency tube and a cyclotron.

### BRIEF DESCRIPTION OF THE DRAWINGS

The present disclosure is further illustrated in terms of exemplary embodiments, and these exemplary embodiments are described in detail with reference to the drawings. These embodiments are not restrictive. In these embodiments, the same number indicates the same structure, wherein:

FIG. 1 is a schematic diagram illustrating an exemplary structure of a radiation therapy device according to some embodiments of the present disclosure;

FIG. 2 is a schematic diagram illustrating an exemplary deflection of a charged particle beam according to some embodiments of the present disclosure;

FIG. 3 is a schematic diagram illustrating an exemplary structure of a focusing magnet according to some embodiments of the present disclosure;

FIG. 4 is a schematic diagram illustrating an exemplary side view of a focusing magnet according to some embodiments of the present disclosure;

FIG. 5 is a schematic diagram illustrating an exemplary side view of a focusing magnet according to some embodiments of the present disclosure;

FIG. 6 is a schematic diagram illustrating an exemplary location of a treatment piece according to some embodiments of the present disclosure;

FIG. 7 is a schematic diagram illustrating another exemplary structure of a radiation therapy device according to some embodiments of the present disclosure;

FIG. 8 is a schematic diagram illustrating an exemplary structure of a target and a multi-leaf collimator according to some embodiments of the present disclosure; and

FIG. 9 is a schematic diagram illustrating an exemplary side view of a target and a multi-leaf collimator according to some embodiments of the present disclosure.

### DETAILED DESCRIPTION

To more clearly illustrate the technical solutions related to the embodiments of the present disclosure, a brief introduction of the drawings referred to the description of the embodiments is provided below. Obviously, the accompanying drawing in the following description is merely some examples or embodiments of the present disclosure, for those skilled in the art, the present disclosure may further be applied in other similar situations according to the drawings without any creative effort. Unless obviously obtained from the context or the context illustrates otherwise, the same numeral in the drawings refers to the same structure or operation.

As used in the disclosure and the appended claims, the singular forms “a,” “an,” and “the” include plural referents unless the content clearly dictates otherwise. Generally speaking, the terms “comprise” and “include” only imply that the clearly identified steps and elements are included, and these steps and elements may not constitute an exclusive list, and the method or device may further include other steps or elements.

A flash radiation therapy (i.e., flash therapy) is a research hotspot in a field of tumor radiation therapy in recent years, which uses an ultra-high dose rate (e.g., greater than 100 Gy/s) to inject all radiation doses into a target area in a short time (e.g., 1-50 ms). An organism may occur flash effect after performing the flash therapy (a sensitivity of tumor tissues to rays still exist while normal tissue is resistant to the rays), the effect can provide better protection to the normal tissue without reducing the effect of radiation therapy on tumor treatment. Therefore, based on a difference in the sensitivity of the tumor tissue and the normal tissue to the ray, the flash therapy has a subversive advantage in the treatment of tumor.

In the current flash therapy, in order to deliver the doses to a tumor target area from multiple angles, devices that can rotate around the object or multiple devices set up around the object may be used, which has the disadvantages of large volume and high cost and is not convenient for promotion.

The embodiments in the present disclosure provide a radiation therapy device, wherein the charged particle beam may be deflected to multiple angles by a scanning magnet, the charged particle beam may incident from a relatively large angle range to the focusing magnet, and the charged particle beam deflected by the focusing magnet may converge to a focus from different angles.

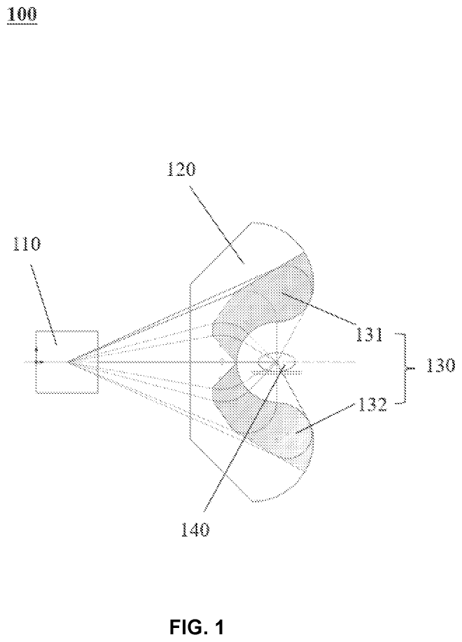

FIG. 1 is a schematic diagram illustrating an exemplary structure of a radiation therapy device 100 according to some embodiments of the present disclosure. As shown in FIG. 1, the radiation therapy device 100 may include a beam generating device, a scanning magnet 110, and one or more focusing magnets 120. The beam generating device may be configured to generate a charged particle beam. The scanning magnet 110 may be configured to diverge the charged particle beam. The divergence refers to a deflection of the charged particle beam that travels in one direction originally to multiple directions, i.e., for deflection at different angles, a point at which the charged particle beam begins to deflect may be set as a deflection start point, a deflection angle may be. The focusing magnet 120 may be configured to converge the charged particle beam deflected by the scanning magnet 110. The convergence refers to injecting the charged particle beam from multiple angles to a same region or at a same point. In some embodiments, when the charged particle beam deflected by the focusing magnet 120 converges at a point, the point may be considered as a treatment center point, a center point corresponding to a focus in a patient 140, or the like. In some embodiments, when the focus in the patient 140 is relatively large, the charged particle beam deflected by the focusing magnet 120 may not converge at a point, but converge a range corresponding to a focus region. In other embodiments, a path of the charged particle beam may be guided by other devices to converge or emit according to treatment needs.

The beam generating device may be configured to generate a charged particle beam. In some embodiments, the beam generating device may include an accelerator. The accelerator may be a device used to accelerate the charged particle beam, which uses a certain form of electromagnetic field to accelerate the charged particle such as a positive electron, a negative electron, a proton, and a heavy ion to a certain energy. In some embodiments, the beam generating device may include but is not limited to a linear accelerator, a cyclotron, an electrostatic accelerator, a synchrotron, a voltage multiplier, a high gradient radio frequency tube, or the like. The cyclotron may be used in proton and heavy ion therapy. In some embodiments, the use of scanning magnet 110 and focusing magnet 120 may make a power density of charged particle beam scattered into multiple angles and irradiate as much area as possible, so that cyclotron may be used in ultra-high energy electron flash therapy.

The scanning magnet 110 may be configured to diverge the charged particle beam. An irradiation field of the charged particle beam after deflecting by the scanning magnet 110 may change, for example, the irradiation field of the charged particle beam may be changed from a point shape to a band shape, which may achieve uniform irradiation within a certain range, and disperse the power density of charged particle beam.

In some embodiments, the scanning magnet 110 may scan the charged particle beam at a certain angle in a certain direction at a certain moment (such as a T1 moment), and scan the charged particle beam at a certain angle in a certain direction at a next moment (such as a T2 moment), and the angles may correspond to a magnetic field of the scanning magnet 110, for example, the magnetic field of the scanning magnet 110 may be strengthened, a deflected angle may be relatively large. In some embodiments, the irradiation field of the charged particle beam may be controlled by controlling a change of the magnetic field of the scanning magnet 110.

In some embodiments, the scanning magnet 110 may be a two-pole magnet or a multi-pole magnet, such as a four-pole magnet, a six-pole magnet, or the like.

The focusing magnet 120 may be configured to deflect the charged particle beam diverged by the scanning magnet. In some embodiments, the charged particle beam deflected by the focusing magnet 120 may converge at a treatment center point after emitting the focusing magnet 120 or converge in a region including the treatment center point. In some embodiments, the focusing magnet 120 may include an entrance and exit, the entrance may be configured for injection of the charged particle beam, and the exit may be configured for emission the charged particle beam. In some embodiments, a deflection angle of the charged particle beam between the entrance and the exit may be 8.

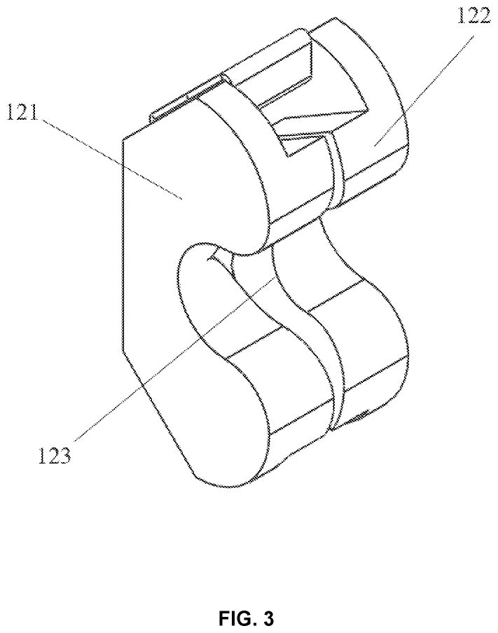

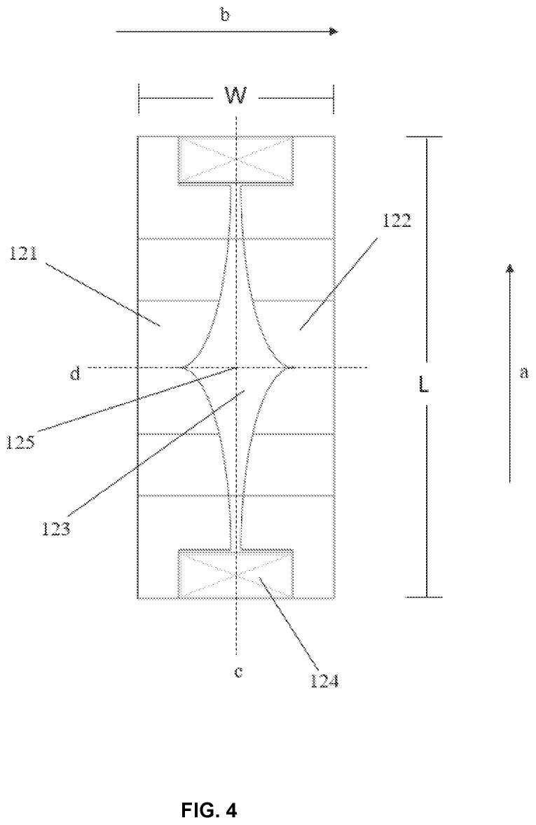

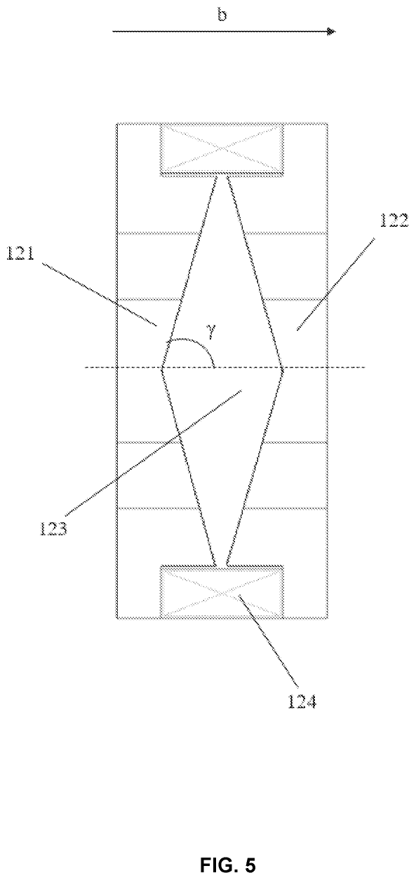

In some embodiments, the focusing magnet 120 may generate a magnetic field, for example, an effective magnetic field region 130 for the deflection of the charged particle beam may be formed and a magnetic field intensity of the generated magnetic field may be not uniformly distributed. For example, a magnetic field intensity near the patient 140 may be lower, a magnetic field intensity away from the patient 140 may be higher, resulting in less deflection for the charged particle beam near the patient 140 and more deflection for the charged particle beam away from the patient 140, which can converge the charged particle beam. In some embodiments, the focusing magnet 129 may include a gap used for passing the charged particle beam, a magnetic field intensity at the gap may be lower relative to that at other positions. More descriptions of the gap of the focusing magnet 120 may be found in FIG. 3, FIG. 4, FIG. 5, and the related descriptions.

In some embodiments, the entrance of the focusing magnet 120 may be set toward the scanning magnet 110, and the exit of the focusing magnet 120 may be set toward the focus in the patient 140. In some embodiments, the charged particle beams passing through the effective magnetic field region 130 may converge at a point. In some embodiments, a center line of the focusing magnet 120, a center line of the scanning magnet 110, and a center point of the focus may be on a same line. A first effective magnetic field region 131 may be above the line and a second effective magnetic field region 132 may be below the line. The particle beam passing through the first effective magnetic field region 131 and the second effective magnetic field region 132 may irradiate the target from upper and lower angles, respectively. If the target only needs to be irradiated from the upper angle or the lower angle, the focusing magnet 120 may include one of the first effective magnetic field region 131 or the second effective magnetic field region 132. In some embodiments, a range of the first effective magnetic field region 131 may be the same as a range of the second effective magnetic field region 132, i.e., the first effective magnetic field region 131 and the second effective magnetic field region 132 are symmetrical with respect to a center line of the focusing magnet 120, as shown in FIG. 1, the obtained irradiation field may also be symmetrical with respect to the center line of the focusing magnet 120. In addition, a range of the first effective magnetic field region 131 may be different from a range of the second effective magnetic field region 132, and the range of the first effective magnetic field region 131 and the range of the second effective magnetic field region 132 may be determined according to the calculation and the focusing magnet 120 may be designed accordingly.

In some embodiments, the focusing magnet 120 may include, but is not limited to, a superconducting magnet, an electromagnet, or the like. In a specific embodiment, the focusing magnet 120 may include at least one of group pairs, the at least one group of coil pairs may generate the effective magnetic field region 130 by applying current to the at least one group of coil pairs.

In some embodiments, the charged particle beam may be affected by Lorentz force and change a motion direction when the charged particle beam moves in the magnetic field after injecting from the entrance of the focusing magnet 120. When the charged particle beam injects the effective magnetic field region 130 of the focusing magnet 120, the charged particle beam may move along an arc and emit from the focusing magnet 120 at multiple angles and converge at the treatment center.

In some embodiments, the charged particle beam deflected by the focusing magnet may converge at the treatment center point within a range where a treatment center point is taken as a center and a central angle is greater than 180° (e.g., 350°, 240°, 195°, etc.).

In some embodiments, a deflection angle of the charged particle beam may be within a range of 0-150°. In some embodiments, the deflection angle of the charged particle beam may be within a range of 0-140°. In some embodiments, the deflection angle of the charged particle beam may be within a range of 20-130°. In some embodiments, the deflection angle of the charged particle beam may be within a range of 20-120°. In some embodiments, the deflection angle of the charged particle beam may be within a range of 40-100°. In some embodiments, the deflection angle of the charged particle beam may be within a range of 50-90°. In some embodiments, the deflection angle of the charged particle beam may be within a range of 60-90°. The deflection angle of the charged particle beam may be affected by the magnetic field intensity distribution of the focusing magnet, the deflection angle of the charged particle beam may be large at a position with high magnetic field intensity, the deflection angle of the charged particle beam may be small at a position with low magnetic field intensity, and the charged particle beam may be not deflected at a position without magnetic field (i.e., the deflection angle may be 0°). In some embodiments, the deflection angle of the charged particle beam injected into a middle of the focusing magnet 120 may be 0°, the deflection angle of the charged particle beam at two ends of the focusing magnet 120 may be 150° or other angles greater than 0°. More descriptions of the middle and the two ends of the focusing magnet 120 and the magnetic field distribution may be found in FIG. 3, FIG. 4, FIG. 5, and the related descriptions.

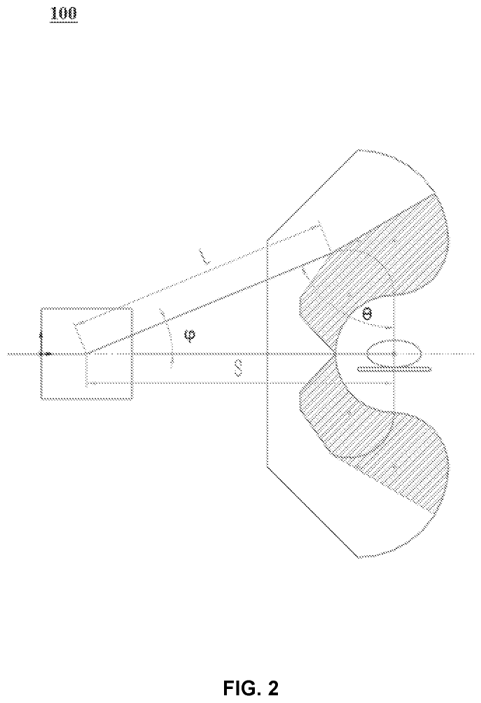

In some embodiments, the scanning magnet 110 and the focusing magnet 120 may be set according to the needs of the effective magnetic field region 130. FIG. 2 is a schematic diagram illustrating an exemplary deflection of a charged particle beam according to some embodiments of the present disclosure. As shown in FIG. 2, a distance between the deflection start point and the entrance of the scanning magnet 110 may be set as L, a distance between the deflection start point of the scanning magnet 110 and the treatment center point may be set as S; a relationship between the scanning magnet 110 and the focusing magnet 120 may meet a following formula: L/(sin(180°−φ−θ)=S/sin θ).

In some embodiments, each focusing magnet 120 may be set to bend towards the exit of each focusing magnet according to the needs of the effective magnetic field region 130. The exit of the focusing magnet 120 may be in a shape of an arc, which may be an approximate arc rather than a strict arc. In some embodiments, the exit of the focusing magnet 120 may be set in other shapes as needed, and the charged particle beam deflected by the focusing magnet 120 may converge in a set region. In some embodiments, the exit of the arc may be centered on the treatment center point, and a corresponding center angle may be greater than 180°. In the arc range, the charged particle beam may be deflected by the focusing magnet 120 and then emit from any position at the arc exit of the focusing magnet 120, and converge at the treatment center point, to perform multi-angle surrounding irradiation on the focus. In some embodiments, the center angle may be an angle between the two particle beams closest to the two ends of the focusing magnet 120 among the rays emitted from the exit. During treatment, the particle beam may pass through the body surface and cause certain damage to the body surface. Therefore, a range of body surface may be increased as much as possible under the given dose rate, which can effectively reduce the damage to body surface per unit area. In some embodiments, after treatment for a period of time, the patient 140 (i.e., a treatment bed) may be reversed from head to tail (e.g., reversing an inward direction and an outward direction), which may achieve 360° treatment easily.

In some embodiments, a center angle corresponding to the arc exit of the focusing magnet 120 may be within a range of 180°-360°. For example, the center angle corresponding to the arc exit of the focusing magnet 120 may be 300°. As another example, the center angle corresponding to the arc exit of the focusing magnet 120 may be 280°. As a further example, the center angle corresponding to the arc exit of the focusing magnet 120 may be 240°.

In some embodiments, a radius of the arc exit of the focusing magnet 120 may be within a reasonable range for ensuring the particle beam perform the bending motion in the arc exit of the focusing magnet 120.

FIG. 3 is a schematic diagram illustrating an exemplary structure of a focusing magnet according to some embodiments of the present disclosure. FIG. 4 is a schematic diagram illustrating an exemplary side view of a focusing magnet according to some embodiments of the present disclosure.

In some embodiments, a length of the focusing magnet 120 may be within a reasonable range. In some embodiments, the length of the focusing magnet 120 may be less than or equal to 4 m. For example, the length of the focusing magnet 120 may be 4 m. As another example, the length of the focusing magnet 120 may be 2 m. As shown in FIG. 4, the length of the focusing magnet 120 refers to a dimension L of the focusing magnet 120 in a direction a.

In some embodiments, a width of the focusing magnet 120 may be within a reasonable range. In some embodiments, the width of the focusing magnet 120 may be less than or equal to 2 m. For example, the width of the focusing magnet 120 may be 2 m. As another example, the width of the focusing magnet 120 may be 1 m. As shown in FIG. 4, the width of the focusing magnet 120 refers to a dimension W of the focusing magnet 120 in a direction b. The direction a is perpendicular to the direction b, the direction a is in a horizontal direction, and the direction b is in a vertical direction.

As shown in FIG. 3, in some embodiments, the focusing magnet 120 may include a first portion 121 and a second portion 122. In some embodiments, the first portion 121 and the second portion 122 may be segmented vertically (i.e., the direction a in FIG. 4), a shape of the first portion 121 may be the same as a shape of the second portion 122, and the first portion 121 and the second portion 122 may be symmetrical along a symmetry axis c of the segment position. In some embodiments, the first portion 121 and the second portion 122 may be made of the same materials. In some embodiments, the shape of the first portion 121 may be not completely the same as the shape of the second portion 122, under this condition, the first portion 121 and the second portion 122 may be separately arranged on both sides of the symmetry axis c, and have different magnetic field shape and intensity, which can satisfy the irradiation of a medical plan on the focus. In some embodiments, a chamber 123 used to install a coil may be arranged between the first portion 121 and the second portion 122, to make the focusing magnet 120 generate the magnetic field. In other embodiments, the focusing magnet 120 also has a magnetic field in other manners, such as using a magnet as a manufacturing material, setting a coil at other positions, etc.

In some embodiments, a gap 123 may be arranged between the first portion 121 and the second portion 122, and the gap 123 may be used to pass the charged particle beam. As shown in FIG. 4, in some embodiments, the focusing magnet 120 may be symmetrical with respect to a symmetry axis d, an intersection point 125 of the symmetry axis d and the symmetry axis c may be considered as a center point of the focusing magnet 120 in FIG. 4. Since the gap 123 is arranged between the first portion 121 and the second portion 122, the intersection point 125 may be located in the gap, the intersection 125 and positions near intersection 125 may be considered to correspond to a middle position of gap 123. In the vertical direction (i.e., the direction a in FIG. 4), a dimension of the middle position of the gap 123 may be greater than a dimension of two ends position of the gap 123, the two ends position of the gap 123 may be considered as positions away from the intersection point 125, and the chamber 124 used to install the coil may be arranged near the two ends position. Since the gap 123 may not generate the magnetic field while the first portion 121 and the second portion 122 may generate the magnetic field, a magnetic field intensity at the middle position of the focusing magnet 120 may be less than a magnetic field intensity at the two ends position of the focusing magnet 120. In some embodiments, a magnetic field intensity at the intersection point 125 may be 0, and the charged particle beam passing through the intersection point 125 may not be deflected. In some embodiments, as shown in FIG. 2, the intersection point 125 may correspond to the treatment center point, that is, the charged particle beam without deflection passing through the intersection point 125 may reach the treatment center point directly. In some embodiments, as shown in FIG. 1, the intersection point 125 may correspond to a position between the first effective magnetic field region 131 and the second effective magnetic field region 132. In some embodiments, the charged particle beam emitted from the two ends position of the gap needs to be deflected at a relatively large angle to converge the charged particle beam at the focus. Therefore, the gap 123 may be set to gradually decrease from the middle position to the two ends position, which makes that the two ends position has a relatively large magnetic field, so that the charged particle beam emitted from the two ends position of the gap 123 has a relatively large deflected angle.

In some embodiments, a difference between the dimension of the middle position of the gap and the dimension of the two ends position of the gap may be within a reasonable range. In some embodiments, the difference between the dimension of the middle position of the gap and the dimension of the two ends position of the gap may be less than or equal to 20 cm. For example, the difference between the dimension of the middle position of the gap and the dimension of the two ends position of the gap may be 20 cm. As another example, the difference between the dimension of the middle position of the gap and the dimension of the two ends position of the gap may be 10 cm.

In some embodiments, a ratio of the dimension of the middle position of the gap 123 to the dimension of the two ends positions of the gap 123 may be within a reasonable range. In some embodiments, the ratio of the dimension of the middle position of the gap 123 to the dimension of the two ends positions of the gap 123 may be less than or equal to 5:1. For example, the ratio of the dimension of the middle position of the gap 123 to the dimension of the two ends positions of the gap 123 may be 5:1. As another example, the ratio of the dimension of the middle position of the gap 123 to the dimension of the two ends positions of the gap 123 may be 3:1.

In some embodiments, a dimension of the gap may gradually decrease from the middle position to the two ends position, an edge of the gap 123 has a smooth trajectory from the middle position to the two ends position, e.g., an arc trajectory shown in FIG. 4.

FIG. 5 is a schematic diagram illustrating an exemplary side view of a focusing magnet according to some embodiments of the present disclosure. In some embodiments, a trajectory of the edge of the gap 123 from the middle position to the two ends positions may be a straight line or an arc, e.g., the arc trajectory shown in FIG. 4, the straight trajectory shown in FIG. 5.

In some embodiments, when the trajectory is the straight line, the straight line has a tilt angle γ relative to the horizontal direction (i.e., the direction b in FIG. 4), and the tilt angle γ may be within a reasonable range, which is related to many variables such as a height of the focusing magnet, etc. In some embodiments, when the trajectory is the arc, a curvature change rate of the arc may be within a reasonable range.

In some embodiments, a count of the focusing magnet 120 may be at least two, the at least two focusing magnets 120 may be arranged adjacently and/or oppositely. When the at least two focusing magnets are arranged oppositely, the exits of the focusing magnets may be opposite. In a specific embodiment, the exits of the two focusing magnets 120 may be strictly oppositely arranged (e.g., center lines of the two focusing magnets 120 being in a same line), or the exits of the two focusing magnets 120 may be roughly oppositely arranged (e.g., an angle between center lines of the two focusing magnets 120 being 140°, 150°, etc.), as long as an emit range of the particle beam from the exits of the focusing magnets 120 covers a range of 360° to realize 360° irradiation therapy for the patient 140. In some embodiments, the at least two focusing magnets 120 may be used and arranged around the patient 140, the exits of the focusing magnets 120 may be all oriented toward the patient 140, which can realize the 360° irradiation therapy for the patient 140. In some embodiments, each focusing magnet may be configured with a beam generating device and a scanning magnet 110, to make the charged beam generated by the corresponding beam generating device emit from the focusing magnets 120. In some embodiments, the at least two focusing magnets 120 may share a same beam generating device and a same scanning magnet 110, which may cause that the particle beam emitted from the exits of the at least two focusing magnets 120 covers the needed range.

The radiation therapy device 100 illustrated in some embodiments of the present disclosure may be used for the ultra-high energy electron flash radiotherapy scheme, i.e., an ultra-high energy electron beam obtained by accelerating electrons through the accelerator may converge the tumor position from multiple angles after passing through the radiation therapy device 100, which can realize the tumor therapy. A range of the ultra-high electron may be 100 MeV-200 MeV, a dose rate may reach 30 Gy/s, and a treatment depth may reach about 15 cm. The radiation therapy device 100 illustrated in some embodiments of the present disclosure may also be used in radiotherapy schemes of other charged particles (e.g., proton). In addition, the radiation therapy device 100 illustrated in some embodiments of the present disclosure may further be applied to other medical schemes as needed.

In some embodiments, the ultra-high energy electron flash therapy system may include a beam generating device, a scanning magnet 110, one or more focusing magnets 120, and/or at least one treatment piece 150; and the beam generating device may include at least one of a high-gradient radio frequency tube and a cyclotron.

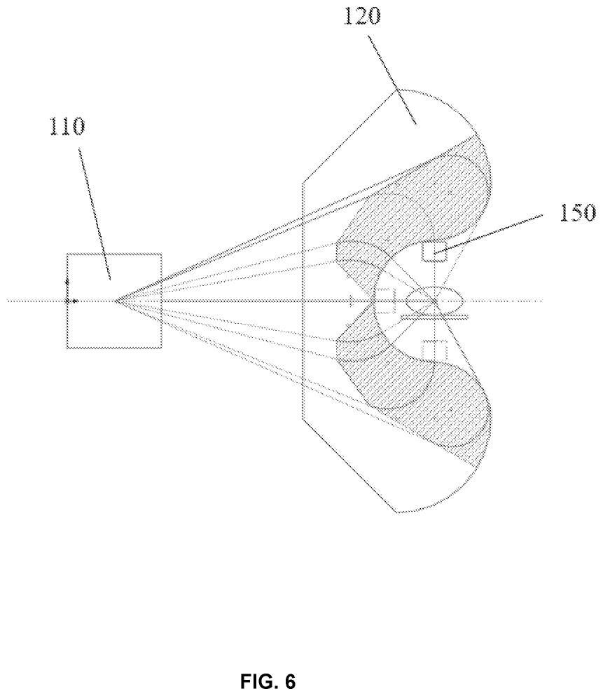

FIG. 6 is a schematic diagram illustrating an exemplary location of a treatment piece 150 according to some embodiments of the present disclosure. In some embodiments, one or more treatment pieces may be movably arranged at the exit of each focusing magnet. As shown in FIG. 6, the one or more treatment pieces may be used to diverge the charged particle beam passing through the focusing magnets 120.

The treatment piece 150 may be configured to guide the charged particle beam to form a predetermined trajectory to make the charged particle beam diverge, and the diverged area may cover a designated region, e.g., a tumor region, thus a treatment with full coverage of tumor area may be realized without moving the treatment bed. In some embodiments, the treatment piece 150 may include a divergent magnet, for example, the divergent magnet may be a combination of a pair of bipolar magnets or multiple magnets that may guide the deflection of the charged particle beam. As another example, the divergent magnet may be a pair of bipolar electromagnets with orthogonal deflection directions. In other embodiments, the treatment piece 150 may include an orthogonal deflection plate using an electric field, and use manners of the orthogonal deflection plate may be similar to the divergent magnet and include similar functions.

In some embodiments, one or more treatment piece 150 may be moved along the exit of the focusing magnet 120, for example, one or more treatment piece 150 may move along an extension direction of an arc exit of the focusing magnet 120. In some embodiments, the one or more treatment pieces 150 may be fixed during the implementation of the irradiation therapy, after completing a stage of the therapy, the position may be changed by moving, and a next stage of the therapy may be continued. In other embodiments, the one or more treatment pieces 150 may be moved according to a medical plan during the irradiation treatment process, so that the charged particle beam may be irradiated to the designated region to complete the treatment according to the medical plan. In some embodiments, when the count of the treatment piece 150 is multiple, positions of multiple treatment pieces 150 may be set according to different medical plans. For example, before starting the therapy, according to the set medical plan, a position of the treatment piece 150 may be determined based on the focus position, the treatment piece 150 may be moved until the treatment piece 150 reaches a set position and remain fixed to start the therapy. In some embodiments, one or more of the multiple treatment pieces 150 may be fixed or movable. For example, the one or more of the multiple treatment pieces 150 may be fixed, and the rest of the multiple treatment pieces 150 may be moved to a set position based on the medical plan as needed, and then start the therapy.

In some embodiments, the one or more treatment pieces 150 may be fixed, for example, the one or more treatment pieces 150 may be arranged at two ends position and/or middle position of the exit of the focusing magnet 120. In a specific embodiment, the exit of the focusing magnet 120 may be provided with two fixed treatment pieces 150. One of the treatment pieces 150 may be in a same line with a center of the scanning magnet 110 and the focusing magnet 120, the other treatment pieces 150 may be arranged at one end of the exit of the focusing magnet 120 and a connecting line between the treatment piece and the treatment center point may be perpendicular to a straight line of a center position of the scanning magnet 110 and the focusing magnet 120. In another specific embodiment, two treatment pieces 150 may be fixed at two ends of the exit of the focusing magnet 120 respectively, and a connecting line of the two treatment pieces 150 may be perpendicular to the straight line of the center position of the scanning magnet 110 and the focusing magnet 120.

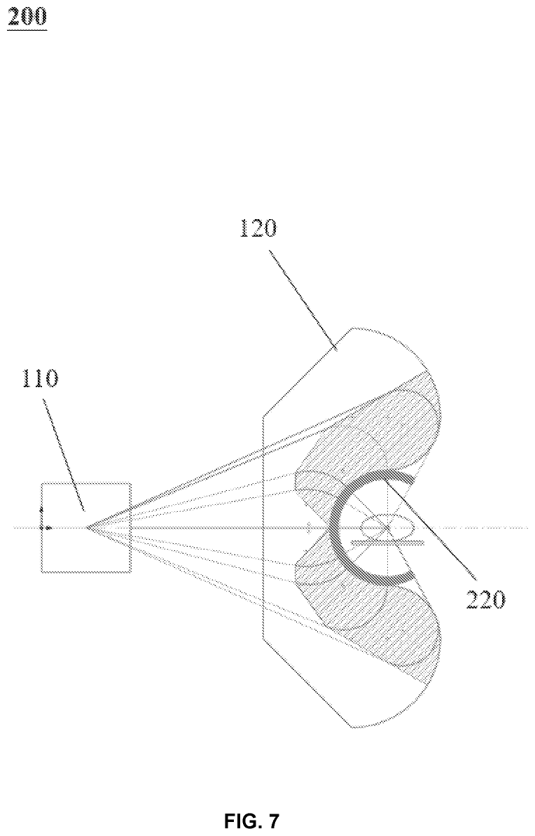

FIG. 7 is a schematic diagram illustrating another exemplary structure of a radiation therapy device 200 according to some embodiments of the present disclosure. The radiation therapy device 200 with another structure illustrated in some embodiments of the present disclosure may be used for a photon flash radiation therapy scheme, i.e., the tumor therapy may be realized by photon irradiation, a photon energy range may be 6 MV-15 MV, a dose rate may be 30 Gy/s, and a treatment depth may be about 15 cm. The radiation therapy device 200 with another structure illustrated in some embodiments of the present disclosure may further be applied to other particle radiotherapy schemes or other medical schemes as needed, which may not be limited herein.

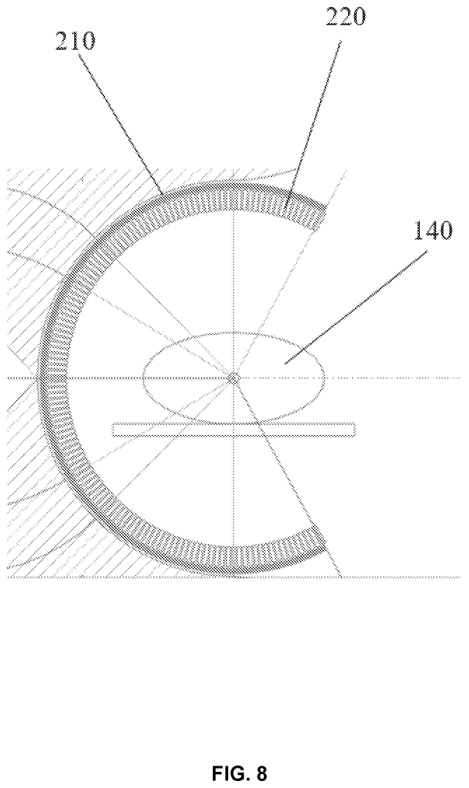

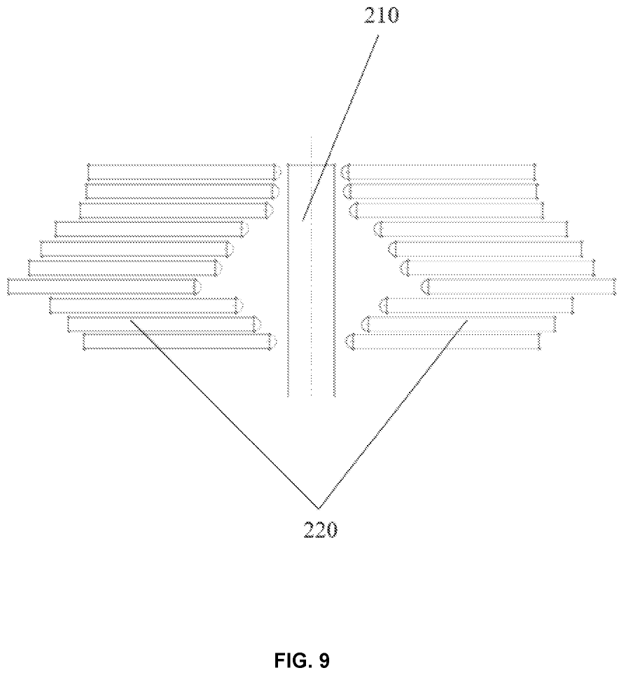

As shown in FIG. 7, in some embodiments, a photon flash therapy system may include a beam generating device, a scanning magnet 110, one or more focusing magnets 120, and/or a target and a multi-leaf collimator; and the beam generating device may include at least one of a petal-shaped accelerator and a cyclotron. The structures and functions of the scanning magnet 110 and the focusing magnet 120 may be similar to the structure and function of the radiation therapy device 100, which may not be limited herein. The beam generating device may be a linear accelerator, an electrostatic accelerator, a synchrotron, a voltage multiplier accelerator, or the like. The cyclotron may be mostly used in proton and heavy ion therapy. Since the petal-shaped accelerator has a high power, the petal-shaped accelerator may be hardly used currently in radiation therapy. However, in some embodiments of the present disclosure, due to the use of the scanning magnet 110 and the focusing magnet 120, a power density of the charged particle beam may be dispersed to irradiate in as many areas as possible from multiple angles, and a limitation that the high-power petal-shaped accelerator is difficult to be used for the radiation therapy may be eliminated. The radiation therapy device 200 may further include a target and a multi-leaf collimator, more descriptions of the target and the multi-leaf collimator may be found in FIG. 8, FIG. 9, and the related descriptions.

FIG. 8 is a schematic diagram illustrating an exemplary structure of a target 210 and a multi-leaf collimator 220 according to some embodiments of the present disclosure. FIG. 9 is a schematic diagram illustrating an exemplary side view of a target 210 and a multi-leaf collimator 220 according to some embodiments of the present disclosure.

In some embodiments, as shown in FIG. 8, the radiation therapy device 200 may include a target 210, and the charged particle beam (e.g., electron beam current) may impact the target 210 to generate the photon. The target 210 may be of various shapes, e.g., straight surface and arc-shaped surface, and the shape of the target 210 needs to match a beam direction of the particle beam, for example, the particle beam may impact the target 210 in a direction perpendicular to a target surface to achieve a better effect. In some embodiments, the target 210 may include a metal target, such as a tungsten target, etc. In some embodiments, the target 210 may be set at the exit of the focusing magnet 120, and the electron beam emitted from the focusing magnet 120 may bombard the target 210 to generate the photon. In some embodiments, the target 210 may be set as an arc, thus a focal spot of the charged particle beam on the arc-shaped target 210 may be not fixed. In addition, by setting an arc-shaped target surface with a relatively larger area than a straight-shaped target surface, the heat bearing per unit area may be relatively reduced. Therefore, the design of the arc-shaped target surface may be conducive to heat dissipation and extend a service life of the target 210.

In some embodiments, the charged particle beam may impact the target 210 to generate the photon beam within a range where the treatment center point is taken as a center and the center angle is greater than 180° (e.g., 190°, 240°, 330°, 350°, etc.). In some embodiments, the target 210 may be set close to the exit of the focusing magnet 120, and an angle and a length of the target 210 may be greater than or equal to an angle and a length of the exit of the focusing magnet 120, as shown in FIG. 5, to make the electron beam emitted from the focusing magnet 120 enable to impact the target 210. In some embodiments, a center angle corresponding to an arc edge of the arc target 210 may be greater than 180°. In a specific embodiment, the center angle corresponding to the arc edge of the arc target 210 may be greater than 240°, and a center angle corresponding to an arc exit of the focusing magnet 120 may also be 240°.

In some embodiments, the radiation therapy device 200 may include a multi-leaf collimator 220, as shown in FIG. 7. The multi-leaf collimator 220 may be a device used to generate a conformal radiation field. In some embodiments, the multi-leaf collimator 220 may be arranged at the exit of the focusing magnet 120 and used to confirm a photon radiation field, to make a contour of the radiation field consistent with the tumor shape as much as possible and reduce the radiation damage to non-tumor regions, as shown in FIG. 9.

In some embodiments, the multi-leaf collimator 220 may be an arc, the multi-leaf collimator 220 may fit set close to the arc target 210, and an angle and a length of the multi-leaf collimator 220 may be approximately equal to the angle and length of the arc target 210, which can conform better to the shape.

In some embodiments, the target 210 may be arranged between the exit of the focusing magnet 120 and the multi-leaf collimator 220, as shown in FIG. 7. The electron beam may impact the target 210 to generate the photon, and then the photon may complete the conformal by the multi-leaf collimator 220.

In some embodiments, the target 210 and the multi-leaf collimator 220 may be movable devices, i.e., when there is no need to perform the photoflash therapy, the target 210 and the multi-leaf collimator 220 may be moved to use the irradiation therapy of the charged particle beam.

In some embodiments, the treatment bed with the patient 140 may be moved in translation, rotation, etc. by setting driving devices, movements of the treatment bed may be performed after implementing treatment for a period of time, and the treatment bed may also be moved continuously during the treatment to realize the 360° omnidirectional treatment for the patient 140.

In some embodiments, the drive device may be set to drive the radiotherapy device 100 (200) to move, for example, the radiotherapy device may rotate around the patient, to realize the 360° omnidirectional treatment for the patient 140. In some embodiments, the driving device may drive the radiation therapy device 100(200) to move and output, to realize the irradiation of the particle beam from one angle to another angle.

Because the tumor is three-dimensional, different regions of the tumor need different treatment doses, and a dose distribution may be uneven. While using the radiation therapy device 100 (200) illustrated in some embodiments of the present disclosure, the radiation therapy device 100(200) may be fixed to make the irradiation angle unchanged, and a dose intensity in the irradiation field may be adjusted, for example, the dose rate of the particle beam may be adjusted by adjusting the count of pulses of the particle beam per unit time. In some embodiments, according to an anatomical relationship between a three-dimensional shape of the lesion and related organs at risk, the particle beam may be assigned to different weights to produce an optimized and uneven intensity distribution in the same irradiation field, so as to reduce the beam flux passing through the organs at risk and increase the beam flux of other portions.

The beneficial effects provided by the radiation therapy device in the embodiments of the present discourse may include but are not limited to: (1) the radiotherapy device including a scanning magnet and a focusing magnet having features of a simple structure, a compact structure, a small volume, and low cost; (2) by designing the focusing magnet, an angle of the particle beam converging in the focus region exceeding 180°, which can reduce a beam dose received by the human body per unit area, thus reducing the harm to the human body, in addition, since the beam contacts the human body in a relatively large area, the devices not needing to rotate around the patient during the treatment, which can reduce a space required for the devices to work; (3) solving problem of heat dissipation and life of the target under high-power electron beam by designing the arc target with a relatively large area; (4) by designing an arc-shaped multi-leaf collimator to facilitate conformal, causing a beam irradiation area consistent with the focus region, reducing the harm to the human body. It should be noted that different embodiments may produce different beneficial effects. In different embodiments, the possible beneficial effects may be any one or combination of the above or any other possible beneficial effects.

The basic concepts have been described. Obviously, for those skilled in the art, the detailed disclosure may be only an example and may not constitute a limitation to the present disclosure. Although not explicitly stated here, those skilled in the art may make various modifications, improvements, and amendments to the present disclosure. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of the specification are not necessarily all referring to the same embodiment. In addition, some features, structures, or features in the present disclosure of one or more embodiments may be appropriately combined.

Moreover, unless otherwise specified in the claims, the sequence of the processing elements and sequences of the present application, the use of digital letters, or other names are not used to define the order of the application flow and methods. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various assemblies described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure and aiding in the understanding of one or more of the various embodiments. However, this disclosure may not mean that the present disclosure object requires more features than the features mentioned in the claims. In fact, the features of the embodiments are less than all of the features of the individual embodiments disclosed above.

In some embodiments, the numbers expressing quantities, properties, and so forth, used to describe and claim certain embodiments of the application are to be understood as being modified in some instances by the term “about,” “approximate,” or “substantially.” Unless otherwise stated, “about,” “approximate,” or “substantially” may indicate a ±20% variation of the value it describes. Accordingly, in some embodiments, the numerical parameters set forth in the description and attached claims are approximations that may vary depending upon the desired properties sought to be obtained by a particular embodiment. In some embodiments, the numerical parameters should be construed in light of the number of reported significant digits and by applying ordinary rounding techniques. Although the numerical domains and parameters used in the present application are used to confirm the range of ranges, the settings of this type are as accurate in the feasible range in the feasible range in the specific embodiments.

Each patent, patent application, patent application publication, and other materials cited herein, such as articles, books, instructions, publications, documents, etc., are hereby incorporated by reference in the entirety. In addition to the application history documents that are inconsistent or conflicting with the contents of the present disclosure, the documents that may limit the widest range of the claim of the present disclosure (currently or later attached to this application) are excluded from the present disclosure. It should be noted that if the description, definition, and/or terms used in the appended application of the present disclosure is inconsistent or conflicting with the content described in the present disclosure, the use of the description, definition and/or terms of the present disclosure shall prevail.

At last, it should be understood that the embodiments described in the disclosure are used only to illustrate the principles of the embodiments of this application. Other modifications may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A radiation therapy device, comprising:
a beam generating device configured to generate a charged particle beam;
a scanning magnet configured to diverge the charged particle beam; and
one or more focusing magnets configured to deflect the charged particle beam diverged by the scanning magnet.

2. The radiation therapy device of claim 1, wherein each focusing magnet includes:
an entrance configured for injection of the charged particle beam; and an exit configured for emission of the charged particle beam.

3. The radiation therapy device of claim 1, wherein the charged particle beam deflected by the focusing magnets converges at a treatment center point within a range where the treatment center point is taken as a center and a central angle is greater than 180°.

4. The radiation therapy device of claim 1, wherein the radiation therapy device further includes one or more targets, and each target is arranged at an exit of each focusing magnet.

5. The radiation therapy device of claim 4, wherein the charged particle beam impacts the target to generate a photon beam within a range where the treatment center point is taken as a center and a central angle is greater than 180°.

6. The radiation therapy device of claim 1, wherein the radiation therapy device further includes a multi-leaf collimator, and the multi-leaf collimator is arc and arranged at the exit of each focusing magnet.

7. The radiation therapy device of claim 1, wherein the exit of each focusing magnet is provided with at least one treatment piece, and the at least one treatment piece is movably arranged at the exit of each focusing magnet.

8. The radiation therapy device of claim 1, wherein a count of the focusing magnets is at least two, the at least two focusing magnets are arranged adjacently or oppositely; and when the at least two focusing magnets are arranged oppositely, the exits of the at least two focusing magnets are opposite.

9. The radiation therapy device of claim 1, wherein a deflection angle of the charged particle beam is within a range of 0-150°.

10. The radiation therapy device of claim 2, wherein each focusing magnet bends towards the exit of each focusing magnet, and the exit of each focusing magnet is arc.

11. The radiation therapy device of claim 1, wherein a magnetic field intensity of a magnetic field generated by the focusing magnets is not uniformly distributed.

12. The radiation therapy device of claim 1, wherein a length of each focusing magnet is less than or equal to 4 m; and a width of each focusing magnet is less than or equal to 2 m.

13. The radiation therapy device of claim 1, wherein each focusing magnet includes a first portion and a second portion, a gap is arranged between the first portion and the second portion, and a dimension of a middle position of the gap is greater than a dimension of two ends position of the gap in a vertical direction.

14. The radiation therapy device of claim 13, wherein a difference between the dimension of the middle position of the gap and the dimension of the two ends position of the gap is less than or equal to 20 cm.

15. The radiation therapy device of claim 13, wherein a ratio of the dimension of the middle position of the gap to the dimension of the two ends position of the gap is less than or equal to 5:1.

16. The radiation therapy device of claim 13, wherein a dimension of the gap gradually decreases from the middle position to the two ends position.

17. The radiation therapy device of claim 16, wherein a trajectory of the gap from the middle position to the two ends position is a straight line.

18. The radiation therapy device of claim 16, wherein a trajectory of the gap from the middle position to the two ends position is an arc.

19. A photoflash therapy system, including a radiation therapy device which comprises:
a beam generating device configured to generate a charged particle beam;
a scanning magnet configured to diverge the charged particle beam; and
one or more focusing magnets configured to deflect the charged particle beam diverged by the scanning magnet;
wherein the photoflash therapy system further comprises a target and a multi-leaf collimator; and
wherein the beam generating device includes at least one of a petal-shaped accelerator and a cyclotron.

20. An ultra-high energy electron flash therapy system, including a radiation therapy device which comprises:
a beam generating device configured to generate a charged particle beam;
a scanning magnet configured to diverge the charged particle beam; and
one or more focusing magnets configured to deflect the charged particle beam diverged by the scanning magnet;
wherein the ultra-high energy electron flash therapy system further comprises at least one treatment piece; and
wherein the beam generating device includes at least one of a high-gradient radio frequency tube and a cyclotron.
